(19) United States
(12) Patent Application Publication
(10) Pub. No.: US 2024/0345032 A1
(43) Pub. Date: Oct. 17, 2024

(54) ULTRA LOW-POWER WIRELESS EMI MEASUREMENT

(71) Applicant: Purdue Research Foundation, West Lafayette, IN (US)

(72) Inventors: Nithin Raghunathan, West Lafayette, IN (US); Na Lu, West Lafayette, IN (US); Vishal Saravade, Kennesaw, GA (US); Enrique Silva, West Lafayette, IN (US)

(21) Appl. No.: 18/681,599

(22) PCT Filed: Aug. 8, 2022
(86) PCT No.: PCT/US2022/039753

§ 371 Date: Feb. 6, 2024

Related U.S. Application Data
(60) Provisional application No. 63/230,366, filed on Aug. 6, 2021.

(51) Int. Cl. G01N 29/12; G01N 29/09; G01N 29/24; G01N 29/32; G01N 29/44

(52) U.S. Cl. CPC G01N 29/12; G01N 29/09; G01N 29/2437; G01N 29/326; G01N 29/44; G01N 2291/0232; G01N 2291/0251; G01N 2291/02881

(57) ABSTRACT

An ultra-low-power wireless impedance measurement system is provided having a sensor group with a piezoelectric sensor, a temperature sensor, an impedance analyzer and a wireless transceiver is to transmit associated data via wireless transmission. Processed temperature and impedance data are transmitted to a server, whereby the processed temperature and impedance data are conveyed to a user via a website.

### TECHNICAL FIELD

The present disclosure relates to industrial sensor systems generally, and, more particularly, to a system and method for low-power sensor operations and communications.

### BACKGROUND

In industrial processes, it is often important to evaluate the cure state or moisture content of a mass of material. This is particularly important in curing concrete. Existing solutions require bulky impedance analysis and/or temperature analysis electronics as well as extensive back-end post processing via mathematical simulation software such as MATLAB. The results, while sometimes accurate, extract a cost in inconvenience and high power consumption.

Before proceeding, it should be appreciated that the present disclosure is directed to a system that may address some of the shortcomings listed or implicit in this Background section. However, any such benefit is not a limitation on the scope of the disclosed principles, or of the attached claims, except to the extent expressly noted in the claims.

Additionally, the discussion of technology in this Background section is reflective of the inventors' own observations, considerations, and thoughts, and is in no way intended to accurately catalog or comprehensively summarize any prior art reference or practice. As such, the inventors expressly disclaim this section as admitted or assumed prior art. Moreover, the identification herein of one or more desirable courses of action reflects the inventors' own observations and ideas, and should not be assumed to indicate an art-recognized desirability.

### SUMMARY

As noted above, it is important in industrial processes to evaluate the cure state or moisture content of a mass of material. While techniques exist to accomplish that end, such solutions are deficient in many respects especially in terms of power consumption and equipment size. To this end, in an aspect of the disclosure, an ultra-low power impedance sensor measurement is provided with a small physical profile and high-performance adaptive logic. This enables the economical, nonintrusive, and accurate measurement of electrical and thermal properties of materials using all electrical, PZT and temperature sensors and the transmission of resultant data using multi-frequency wireless protocols such as BLUETOOTH, ANT, LORA, etc. The system can be used to monitor structural health of civil engineering structures using PZT sensors during construction and after the concrete has cured. The circuit can also measure temperatures of the concrete with, e.g., 1-degree accuracy.

While a hub-centered architecture as discussed herein may be employed, an LTE-enabled architecture may be used alternatively for increased efficiency when communicating data to external entities. Reducing the amount of data that must be transferred over LTE in this embodiment assists in conserving battery life and reducing LTE data transfer costs. Example techniques to reduce data load and conserve battery life include careful timing of sweeps and data collection as well as strategically implementing sleep/wake states.

Further, either manual or automatic starting may be used for data collection activities. In an embodiment, an autostart feature is used to trigger the system to automatically start measuring frequency data upon detecting that concrete is poured on the sensor. This embodiment has the beneficial effect of eliminating or reducing the use of power switches. In a further embodiment, the autostart system includes a restart feature to enable an operator or management system to restart a sensor that has been unplugged or otherwise subjected to an unplanned stoppage during operation after an autostart.

In addition to the data collection ability of the system, embodiments of the same equipment are used to harvest energy from high traffic environments, thus maintaining communication ability for longer times. The system also features onboard logic in a further embodiment to establish concrete strengths, negating the need for extensive post-processing.

To these ends, an ultra-low-power wireless impedance measurement system is provided in an aspect of the disclosed principles, the ultra-low-power wireless impedance measurement system including a sensor group having a piezoelectric sensor, a temperature sensor and a measurement module. The measurement module includes an impedance analyzer configured to receive frequency data from the piezoelectric sensor and generate impedance data and a wireless protocol processor. The wireless protocol processor is configured to receive the impedance data from the impedance analyzer, receive temperature data from the temperature, and format the received data for wireless transmission. An included short-range wireless transceiver is configured to receive the formatted data and transmit the data via short-range wireless transmission.

In a further aspect, a short-range wireless hub is provided and is configured to receive the data transmitted by the short-range wireless transceiver of the sensor group. The short-range wireless hub includes a short-range wireless transceiver, a small computing device configured to receive and process an output of the short-range wireless transceiver to produce processed temperature and impedance data, and an internet connection configured to receive the processed temperature and impedance data and transmit the processed temperature and impedance data to a server, whereby the processed temperature and impedance data are conveyed to a user via a website.

These and other aspects and features of the present disclosure will be better understood when read in conjunction with the accompanying drawings.

### BRIEF DESCRIPTION OF THE DRAWINGS

FIG. 1 is a schematic diagram showing an example architecture in accordance with an aspect of the present disclosure;

FIG. 2 is a schematic diagram showing further example architecture in accordance with an aspect of the present disclosure;

FIG. 3 is a flow chart showing a process of hub operation in accordance with an aspect of the present disclosure; and

FIG. 4 is a flow chart showing a process of sensor side device start up and operation in accordance with an aspect of the present disclosure;

FIG. 5 is a flow chart showing an aspect of sensor side device start up and operation in accordance with an aspect of the present disclosure; and

FIG. 6 is a flow chart showing another aspect of sensor side device start up and operation in accordance with an aspect of the present disclosure.

While the following detailed description will be given with respect to certain illustrative embodiments, it should be understood that the drawings are not necessarily to scale and the disclosed embodiments are sometimes illustrated diagrammatically and in partial views. In addition, in certain instances, details which are not necessary for an understanding of the disclosed subject matter or which render other details too difficult to perceive may have been omitted. It should therefore be understood that this disclosure is not limited to the particular embodiments disclosed and illustrated herein, but rather to a fair reading of the entire disclosure and claims, as well as any equivalents thereto. Additional, different, or fewer components and methods may be included in the systems and methods.

### DETAILED DESCRIPTION

In the following description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

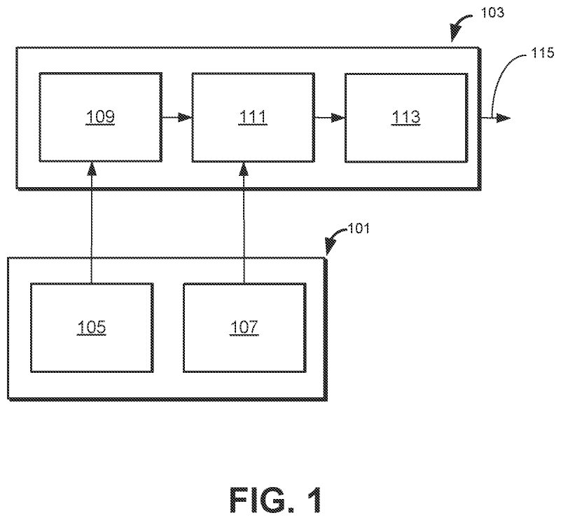

Referring now to the drawings and with specific reference to FIG. 1, this figure shows an example architecture in accordance with an aspect of the present disclosure. The illustrated system includes a sensor group 101 and an EMI measurement device 103. The sensor group 101 in the illustrated architecture includes a PZT sensor 105 and a temperature sensor 107. The PZT sensor 105 is a piezoelectric sensor while the temperature sensor 107 may be a thermocouple or other suitable temperature sensor.

The raw readings from the sensors 105, 107 are sent to the EMI measurement device 103. At the EMI measurement device 103, the data from the PZT sensor 105 is processed by an impedance analyzer 109, and is then passed to a wireless protocol processor 111 such as the NRF52840 or any other suitable processor. The data from the temperature sensor 107 is received directly by the wireless protocol processor 111.

The sensor data received by the wireless protocol processor 111 is then transmitted via a short-range wireless transceiver 113 such as a Bluetooth Low Energy (BLE) or LoRa (spread spectrum) transceiver. The short-range wireless transceiver 113 may be a separate entity or may be part of another system such as the wireless protocol processor 111. In an alternative embodiment, an LTE-enabled architecture is used rather than a short range hub-centered architecture.

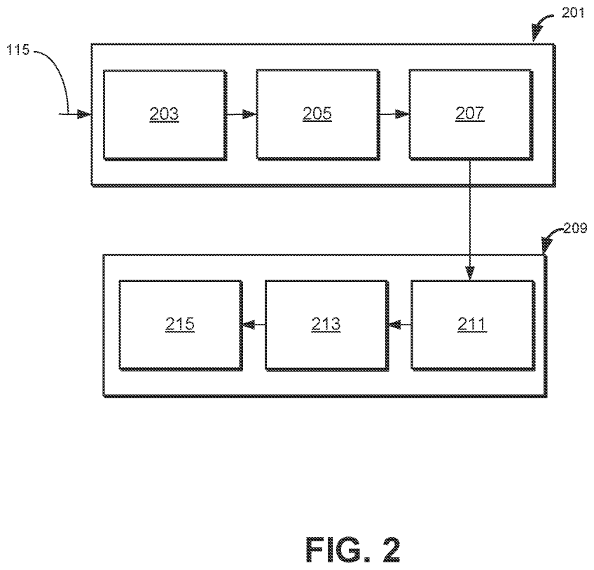

Turning to FIG. 2, in an embodiment employing a hub-centered architecture, the data 115 transmitted by the wireless transceiver 115 is received by a short-range wireless hub 201, such as a BLE (or LoRa) hub. At the short-range wireless hub 201, the received data are processed by an appropriate short-range wireless transceiver 203. From there, the data are provided to a small computing device 205 such as a RASPBERRY PI 3 board for further processing. Finally, the processed data are handed off to an internet connection 207 for transmission to a server 209. It will be appreciated that although the RASPBERRY PI 3 board is used here as an example of a computing device, any other suitable computing device may instead be used.

As such, laptops, personal computers, hand-held computers and other computing systems are included within the scope of this technology. Moreover, in an LTE-enabled architecture, the transmitted data from the sensor group may be sent directly to the server 109.

Within the server 209, the received data are stored in a database 211 and then processed by a data processing module 213 to produce user-required data. The user-required data are then configured in a website accessible to the user of the system, e.g., an industrial technician or operator. In this way, personnel can easily see the required data on any device capable of viewing the website, e.g., a laptop, smartphone, and so on.

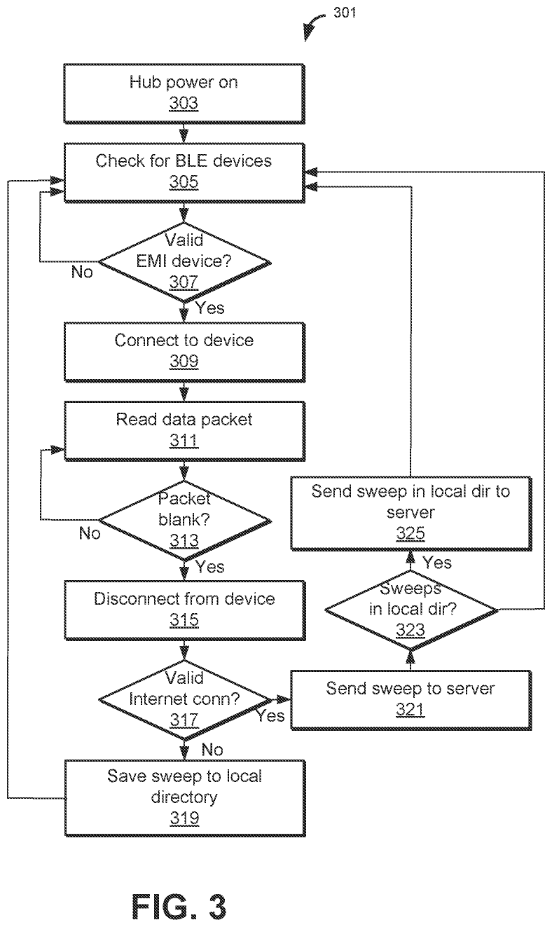

Turning to FIG. 3, this figure shows a flowchart of a process 301 for hub operation in accordance with hub-based embodiments of the present disclosure. At stage 303 of the process 301, the hub is turned on. The hub checks for BLE devices, for example, at stage 307 and returns to stage 305 if none are found. Otherwise, the process 301 moves to stage 309, wherein the hub connects to the found device.

The hub reads a data packet from the device at stage 311, and if the packet is not blank, as determined at stage 313, the process returns to stage 311 to continue reading. Otherwise, the process 301 moves forward to stage 315 and disconnects from the found device. The hub next checks for a valid internet connection at stage 317. If no valid connection is found, the sweep is saved locally and the process 301 returns to stage 305 to check for BLE devices. If instead, a valid internet connection is found, the hub sends the sweep to the server at stage 321, checks for sweeps in the local directory at stage 323, sends any such sweeps at stage 325, and then returns to stage 305 to check for BLE devices.

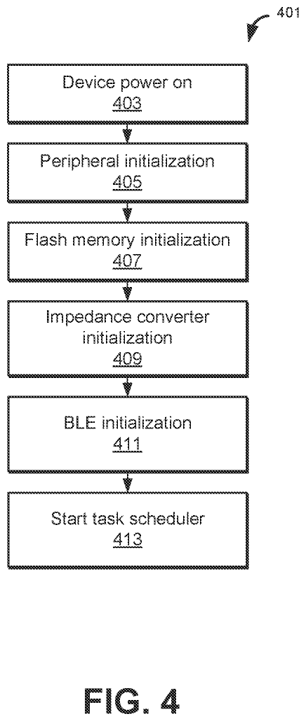

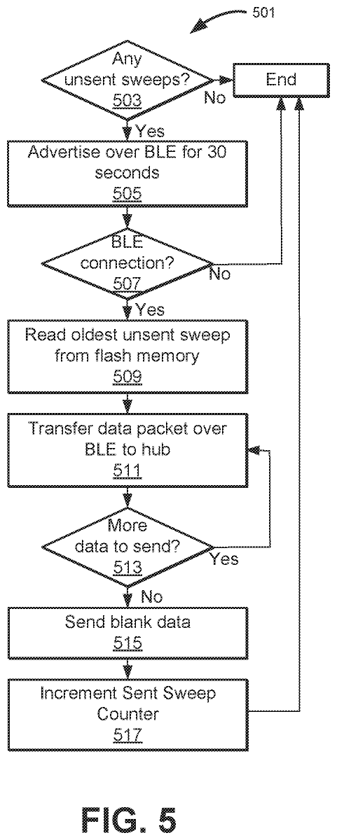

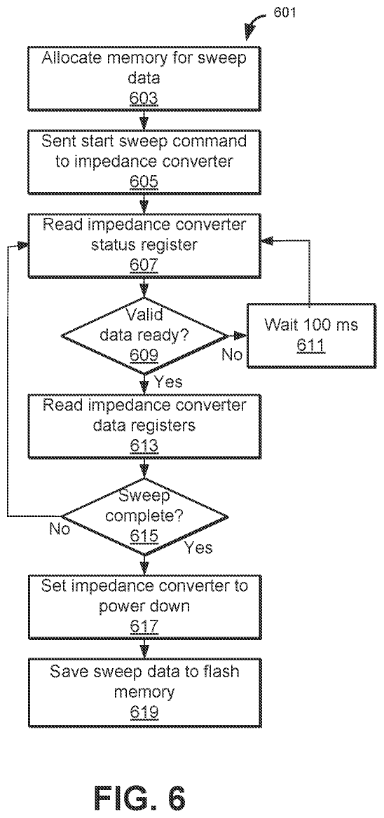

Turning to FIG. 4, this figure, in conjunction with FIGS. 5 and 6, shows a process of sensor side device start up and operation. At stage 403 of the process 401, the device is powered on and the process 401 executes device initialization in stages 405 to 411. At stage 405, peripheral initialization is executed, followed by flash memory initialization at stage 407, impedance converter initialization at stage 409, and BLE initialization at stage 411. After device initialization, the process 401 flows to stage 413, to start a task scheduler process.

The task scheduler triggers a sweep task at a first interval, e.g., every 30 minutes, and triggers a BLE task at a second interval, e.g., every 5 minutes. The flowchart of FIG. 5 shows a process of executing the BLE task. At stage 503 of the process 501, it is determined whether there are any unsent sweeps. If there are not, the process 501 terminates. Otherwise, the process 501 moves to stage 505 wherein it advertises over BLE for 30 seconds. At stage 507, it is determined whether there is a BLE connection. If not, the process 501 terminates. Otherwise, the process 501 moves to stage 509 to read the oldest unsent sweep from flash memory.

Following stage 509, the process 501 moves to stage 511 to transfer the data packet over BLE to the hub, and then checks at stage 513 whether there is more data to send. If so, the process returns to stage 511. Otherwise, the process 501 moves to stage 515 and sends blank data to indicate that the transfer is complete. At stage 517, a Sent Sweep Counter is incremented.

As noted above, in an LTE-enabled rather than hub-based architecture, the data collection and monitoring operations may be spaced and/or timed to reduce power usage and transmission requirements. Relevant techniques include careful timing of sweeps and data collection as well as strategically implementing sleep/wake states. In this way, the higher power requirements of longer range communications need not unduly impact battery life, and data transfer volumes, and hence transfer costs, can be minimized.

Turning to FIG. 6, this figure shows a process of executing the sweep task. At stage 603 of the process 601, the process 601 allocates memory for sweep data, and then sends a start sweep command to the impedance converter at stage 605. The impedance converter status register is read at stage 607. If valid data are not yet ready, as determined at stage 609, the process 601 waits for 100 ms and returns to 607. Otherwise, the impedance converter data registers are read at stage 613. If the sweep is not yet complete, as determined at stage 615, the process returns to stage 607, and otherwise the impedance converter is set to power down at stage 617 and the sweep data are saved to flash memory at stage 619.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more embodiments, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as an “aspect” may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as a “configuration” may refer to one or more configurations and vice versa.

The word “exemplary” is used herein to mean “serving as an example, instance, or illustration.” Any embodiment described herein as “exemplary” or as an “example” is not necessarily to be construed as preferred or advantageous over other embodiments. Furthermore, to the extent that the term “include,” “have,” or the like is used in the description or the claims, such term is intended to be inclusive in a manner similar to the term “comprise” as “comprise” is interpreted when employed as a transitional word in a claim. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

Reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub combination or variation of a sub combination.

What is claimed is:

1. An ultra-low-power wireless impedance measurement system comprising:
a sensor group, the sensor group including:
a piezoelectric sensor;
a measurement module, the measurement module including: an impedance analyzer configured to receive frequency data from the piezoelectric sensor and generate impedance data;
a wireless protocol processor configured to receive the impedance data from the impedance analyzer and to format the received data for wireless transmission; and
a wireless transceiver configured to receive the formatted data and transmit the data via wireless transmission.

2. The ultra-low-power wireless impedance measurement system in accordance with claim 1, wherein the wireless transceiver is an LTE transceiver.

3. The ultra-low-power wireless impedance measurement system in accordance with claim 1, further comprising a temperature sensor for producing temperature data, and wherein the wireless protocol processor is further configured to receive temperature data from the temperature sensor.

4. The ultra-low-power wireless impedance measurement system in accordance with claim 1, wherein the wireless transceiver is a short-range wireless transceiver.

5. The ultra-low-power wireless impedance measurement system in accordance with claim 3, wherein the temperature sensor is a thermocouple device.

6. The ultra-low-power wireless impedance measurement system in accordance with claim 4, wherein the short-range wireless transceiver is a BLE/LoRa transceiver.

7. The ultra-low-power wireless impedance measurement system in accordance with claim 4, wherein the short-range wireless transceiver is integrated with another component of the system.

8. The ultra-low-power wireless impedance measurement system in accordance with claim 4, wherein the short-range wireless transceiver is a stand-alone component.

9. The ultra-low-power wireless impedance measurement system in accordance with claim 4, further comprising a short-range wireless hub configured to receive the data transmitted by the short-range wireless transceiver of the sensor group, the short-range wireless hub comprising:
a short-range wireless transceiver;
a small computing device configured to receive and process an output of the short-range wireless transceiver to produce processed temperature and impedance data; and
an internet connection configured to receive the processed temperature and impedance data and transmit the processed temperature and impedance data to a server, whereby the processed temperature and impedance data are conveyed to a user via a website.

10. The ultra-low-power wireless impedance measurement system in accordance with claim 1, wherein the wireless transceiver is configured to transmit the formatted data to a server, whereby the processed temperature and impedance data are conveyed to a user via a website.

11. The ultra-low-power wireless impedance measurement system in accordance with claim 10, wherein the server is configured convey the processed temperature and impedance data to the user via the website by first storing the processed temperature and impedance data in a database and configuring the processed temperature and impedance data for display via the website.
